(19) United States
(12) Patent Application Publication
(10) Pub. No.: US 2019/0175795 A1
(43) Pub. Date: Jun. 13, 2019

(54) BODY-INSERTABLE MEDICAL CABLE WITH ANTIMICROBIAL CONDUITS AND FILL REGIONS

(71) Applicant: EOS Surfaces, LLC, Norfolk, VA (US)

(72) Inventor: Kenneth G. Trinder, II, Norfolk, VA (US)

(21) Appl. No.: 16/210,622

(22) Filed: Dec. 5, 2018

Related U.S. Application Data
(60) Provisional application No. 62/596,341, filed on Dec. 8, 2017.

(51) Int. Cl. A61L 29/16; H01B 7/04; H01B 3/46; H01B 3/30; H01B 3/42; H01B 3/10; H01B 3/44; G02B 6/44; A61B 1/012; A61B 1/00; A61L 29/08; A61L 29/10; A61B 1/07; A61B 1/005; A61L 29/12

(52) U.S. Cl. CPC A61L 29/16; A61L 2300/102; H01B 3/46; H01B 3/302; H01B 3/427; H01B 3/10; H01B 3/445; G02B 6/443; A61B 1/012; A61B 1/00071; A61L 29/085; A61L 29/106; A61B 1/00114; A61B 1/07; A61B 1/0057; A61L 29/126; A61L 2300/404; A61L 2300/606; A61L 2420/04; H01B 7/048

(57) ABSTRACT

A flexible body-insertable cable includes conduits and cables. Each conduit has an antimicrobial material defining interior wall surfaces thereof. Each cable provides a function selected from the group consisting of mechanical functions, electrical functions, and optical functions. A solid antimicrobial material encases each of the conduits and each of the cables for retaining the conduits and cables in a spaced-apart and longitudinally-extending arrangement along a length of the solid antimicrobial material.

Pursuant to 35 U.S.C. §119, the benefit of priority from provisional application 62/596,341, with a filing date of Dec. 8, 2017, is claimed for this nonprovisional application.

### FIELD OF THE INVENTION

The invention relates generally to body-inserted medical cables, and more particularly to a flexible body-insertable medical cable having antimicrobial conduits and fill regions for use in a variety of medical procedures.

### BACKGROUND OF THE INVENTION

Many invasive medical diagnostic and treatment procedures depend on the use of a body-insertable flexible cable that couples a procedure-based tip to a control system. Typically, the tip includes an optical scope to provide imaging capability and one or more ports to provide egress for tool(s), air, and/or liquids such as water, saline, etc. Such cables typically include a flexible hollow casing that supports a loose collection of narrow mechanical cable-manipulation lines, optical fibers and/or bundles thereof, electrical signal carrying lines, and conduits to support movement of tools and fluids along the length of the flexible cable. As is known in the art, the combination of the flexible hollow casing and its contents to include the tip is generally referred to as a “scope” regardless of the particular medical procedure they will be used to perform. By way of non-limiting examples, such scopes are used in the performance of colonoscopies, sigmoidoscopies, endoscopies, enteroscopies, and percutaneous endoscopic gastrostomy procedures.

The costs associated with medical scopes require their reuse as opposed to being single-use and disposable. To avoid spreading infection, reusable scopes require appropriate reprocessing between uses. Reprocessing involves disinfection and sometimes sterilization steps that vary depending on the use of the scope, the status of the patients, the cleaning protocols of the facility, and the quality of the workers engaged in the reprocessing. Accordingly, failures in reprocessing can occur at many points during a cleaning procedure resulting in contaminated devices that have been associated with outbreaks of hospital-acquired infections at rates higher than those associated with any other medical device. Studies show that these infections can occur even when the scopes are reprocessed according to guidelines provided by the scope's manufacturer and supported by hospital protocols. Failures during scope reprocessing are due to one or a combination of the following four factors:
lapses in reprocessing techniques;
contaminated automatic endoscope reprocessors (AERs);
use of damaged scopes; and/or
flawed scope designs.

The complex structure of cables, fibers/lines, and conduits within a flexible hollow casing of a scope are exposed to a patient's bodily fluids via the scope's tip leading to the possibility of contamination anywhere along the scope and it's interior. Reprocessing steps of cleaning, high-level disinfection, rinsing, and storing are designed to eliminate these contaminated fluids from the many internal crevices within the hollow casing in order to render the scope safe for the next patient. Unfortunately, scopes are hard to clean and their regular use leads to microscopic and visible scratches, cracks, tears, and roughness that can easily collect biological matter to include biofilms. Such contamination exposes future patients to a high risk of acquiring a hospital-associated infection. Furthermore, the design of current flexible scopes makes them uniquely susceptible to bacterial contamination that persists after high-level disinfection. The elements that allow a scope to perform, namely, the cables, fibers/lines and conduits within the flexible hollow casing, are the same elements that allow it to become contaminated with pathogenic organic material.

### SUMMARY OF THE INVENTION

Accordingly, it is an object of the present invention to provide a flexible medical cable for use and reuse in invasive medical procedures.

Another object of the present invention is to provide a flexible medical cable having minimal infectious growth sites.

Still another object of the present invention is to provide a flexible medical cable that has inherent disinfection properties.

Other objects and advantages of the present invention will become more obvious hereinafter in the specification and drawings.

In accordance with the present invention, a flexible body-insertable cable includes a plurality of conduits and a plurality of cables. Each conduit has an antimicrobial material defining interior wall surfaces thereof. Each of the cables provides a function selected from the group consisting of mechanical functions, electrical functions, and optical functions. A solid antimicrobial material encases each of the conduits and each of the cables for retaining the conduits and cables in a spaced-apart and longitudinally-extending arrangement along a length of the solid antimicrobial material. A sheath can be provided to encase the solid antimicrobial material.

### BRIEF DESCRIPTION OF THE DRAWINGS

Other objects, features and advantages of the present invention will become apparent upon reference to the following description of the preferred embodiments and to the drawings, wherein corresponding reference characters indicate corresponding parts throughout the several views of the drawings and wherein:

FIG. 1 is a schematic cross-sectional view of a flexible body-insertable cable in accordance with an embodiment of the present invention;

FIG. 2 is a schematic cross-sectional view of a flexible body-insertable cable in accordance with another embodiment of the present invention;

FIG. 3 is a schematic cross-sectional view of a flexible body-insertable cable in accordance with still another embodiment of the present invention; and

FIG. 4 is a schematic cross-sectional view of a flexible body-insertable cable in accordance with yet another embodiment of the present invention.

### DETAILED DESCRIPTION OF THE INVENTION

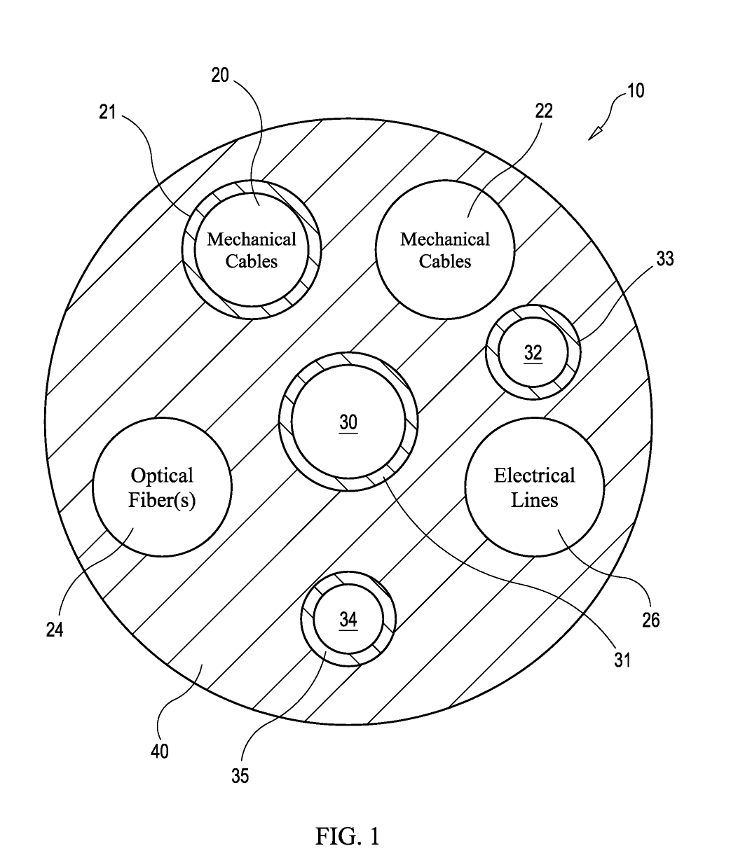

Referring now to the drawings and more particularly to FIG. 1, a schematic cross-sectional view of a body-insertable cable in accordance with an embodiment of the present invention is shown and is referenced generally by numeral 10. Cable 10 includes a number of structural features that are relevant to a medical procedure in which cable 10 will be used. However, it is to be understood that the herein shown/described structural features related to a medical procedure are merely for purposes of illustration and are not limitations of the present invention. That is, the novel features of flexible body-insertable cable in accordance with the present invention can be adapted for use with a variety of body-insertable cables designed for use with a variety of medical scopes. Accordingly, the cable of the present invention is not limited by the design of a scope's control system or tip.

In general, the entirety of cable 10 that is to be inserted into a patient's body (not shown) must be flexible and include the features that will be described herein. Accordingly, it is sufficient to illustrate a cross-sectional view of cable 10 in order to illustrate its novel features. Cable 10 is illustrated to clearly show its features and is not drawn to scale such that the actual size of cable 10 and its internally-housed features are not limitations of the present invention.

In the illustrated embodiment, cable 10 includes mechanical cables 20 and 22, optical fiber(s) 24, and electrical lines 26. As would be understood in the art of medical scopes, multiple sets of mechanical cables 20/22, optical fiber(s) 24, and/or electrical lines 26 could be incorporated in cable 10 without departing from the scope of the present invention. The illustrated positions of the various cables, fibers, and/or lines within cable 10 are for purposes of description only and, as such, are not limitations of the present invention.

Mechanical cables 20 are used to manipulate a procedure-specific tip (not shown) coupled to an end of cable 10 and mechanical cables 22 provide strength along the length of cable 10. In terms of manipulation of a procedure-based tip coupled to cable 10, mechanical cables 20 need to be able to move longitudinally along/within cable 10. A variety of mechanical cables to include shape memory alloy-based cables could be used without departing from the scope of the present invention. For mechanical cables 22 employed as strength members, no such longitudinal movement along/within cable 10 is required. Optical fiber(s) 24 are used to carry light and/or images along the length of cable 10. Electrical lines 26 are used to carry electrical signals along the length of cable 10. In general, optical fiber(s) 24 and electrical lines 26 need not move longitudinally along/within cable 10. Also defined within cable 10 are a number of hollow, tubular conduits such as conduits 30, 32 and 34. More or fewer hollow conduits can be provided without departing from the scope of the present invention. Conduits 30, 32 and 34 are used to support tool and/or fluid movement along the length of cable 10.

All of the above-described cables/fibers/lines and conduits extend along the length of cable 10 and are retained in spaced-apart relationships. For example, all of the cables/fibers/lines and conduits can be parallel to one another. In accordance with the present invention, the spaced-apart relationships are established and maintained by a flexible and solid antimicrobial encasement 40. In the illustrated embodiment, encasement 40 is circular in its outer cross-sectional profile. However, it is to be understood that the outer cross-sectional profile of encasement 40 can be other than circular without departing from the scope of the present invention.

Encasement 40 can be fabricated to be in direct contact with mechanical cables 22, optical fiber(s) 24, and electrical lines 26 since none of these elements are required to move longitudinally along/within cable 10. For mechanical cables 20 that need to move longitudinally along/within cable 10, an antimicrobial hollow tube 21 defines a conduit having interior wall surfaces that can come into contact with mechanical cables 20 as they are moved longitudinally along/within cable 10. With respect to conduits 30, 32 and 34, encasement 40 is in direct contact with antimicrobial tubes 31, 33 and 35, respectively, each of which defines the interior wall surfaces of respective conduits 30, 32 and 34. Encasement 40 could be fabricated using extrusion or molding techniques without departing from the scope of the present invention.

The antimicrobial materials used for tubes 21/31/33/35 and encasement 40 are flexible polymeric materials impregnated with a biocidal material such as a noble metal and alloys thereof (e.g., copper, copper alloys, copper salts, zinc salts, silver salts, and mixtures thereof). By way of an illustrative example, one such biocidal material is cuprous oxide (i.e., Cu₂O). The polymeric materials that are impregnated with a biocidal material can include polyurethane, thermoplastic silicone-polyurethane copolymers (“SPUR”), perfluoroethylene propylene copolymer (“FEP”), polytetrafluoroethylene (“PTFE”), thermoplastic polyurethanes (“PUR”), and polyether ether ketone (“PEEK”). Different polymeric materials can be used in a single cable 10. For example, if conduit 30 is to be used to support the transport of a procedural tool (and its control line), the polymer material used as the base material for antimicrobial tube 31 can be PTFE owing to its toughness such that it will not be easily scratched as a tool and its control line are pushed/pulled there through. The same biocidal-impregnated PTFE material can be used for antimicrobial tube 21 in its support of longitudinally-moving mechanical cables 20. If conduits 32 and 34 are to be used to transport fluids such as air or water, the polymer material used for tubes 33 and 35 can be PUR, SPUR or PEEK since these materials are compatible with the fluids passed there through and can offer flexibility or columnar stiffness and torsional strength while being biocompatible and heat resistant. A suitable choice for a base polymeric material for antimicrobial encasement 40 is FEP because it can be fabricated in long continuous lengths using melt processing techniques.

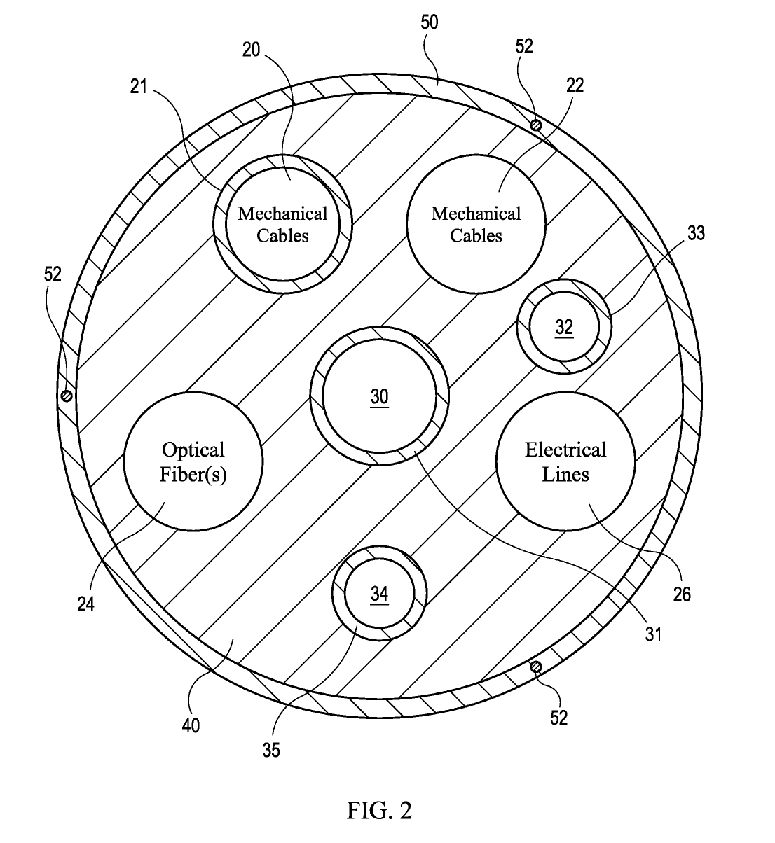

Referring now to FIG. 2, the above-described cable structure can be encased in an outer flexible sheath 50 that can also be a flexible polymeric material impregnated with a biocidal material such that sheath 50 is also considered to be antimicrobial. For example, the material used for sheath 50 can be polyurethane impregnated with a biocidal material such as cuprous oxide. Polyurethane is a suitable choice for the base material of sheath 50 because it has excellent mechanical properties and is biocompatible. Sheath 50 can also have one or more strength members 52 embedded therein as would be understood in the art.

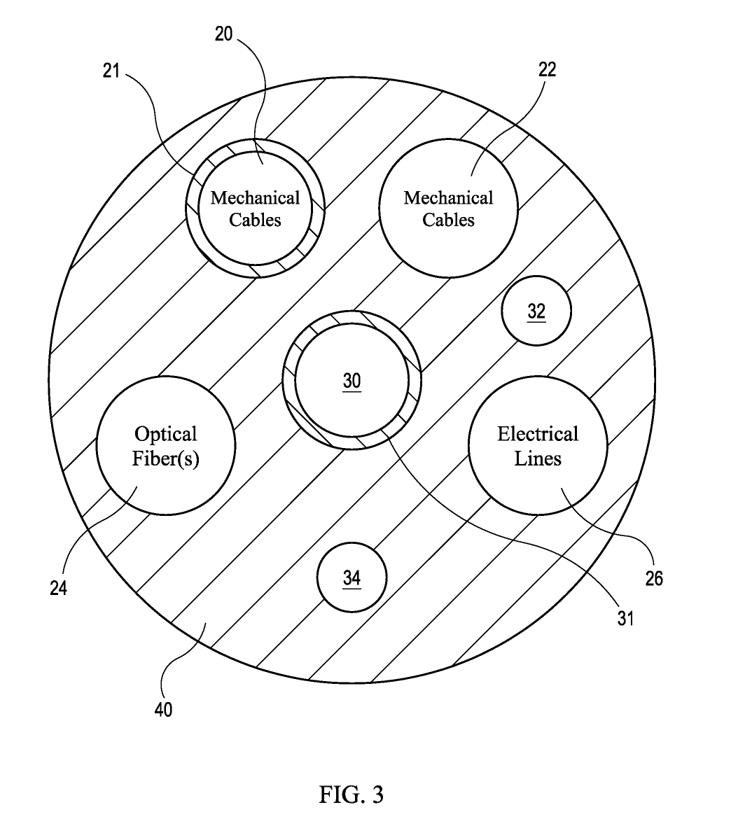

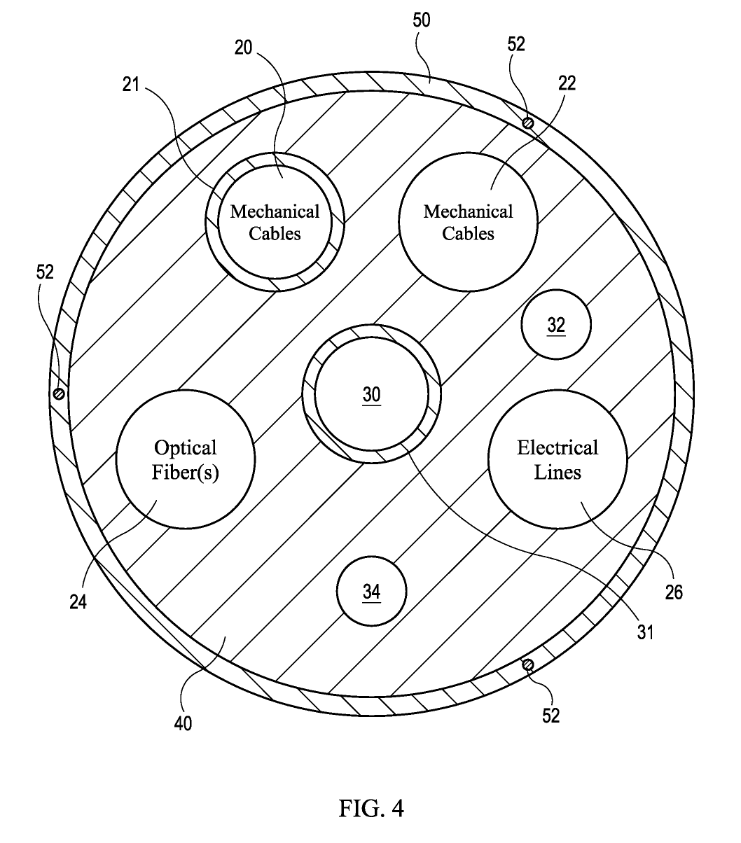

Another embodiment of the present invention is illustrated in FIG. 3 where conduits 32 and 34 have interior wall surfaces defined by the material used for encasement 40. That is, fluid-carrying conduits 32 and 34 and their interior wall surfaces are formed integrally with encasement 40 during the fabrication of encasement 40. The cable structure illustrated in FIG. 3 could also be encased in a flexible outer sheath 50 as shown in FIG. 4. As described previously herein, sheath 50 can be made from a flexible antimicrobial material and can have strength member(s) 52 embedded therein.

The advantages of the present invention are numerous. The flexible body-insertable cable eliminates the many crevices present in existing body-insertable cables to thereby greatly reduce infection-susceptible sites along and within such a cable. Furthermore, all surfaces of the cable that will be or might be exposed to bodily fluids are antimicrobial to provide constant infection control and prevention. By lining the tool-carrying conduit of the cable with a scratch-resistant antimicrobial material, the cable reduces the creation of infection-susceptible sites within the cable. However, even if the interior wall surfaces of the various conduits do become scratched, the resulting scratched interior surfaces increase the overall surface area of the interior wall surfaces to effectively increase the inherent infection-fighting properties of the antimicrobial materials. For all of the above reasons, the flexible body-insertable cable of the present invention will drastically reduce or eliminate infection problems associated with reusable medical scopes.

Although the invention has been described relative to a specific embodiment thereof, there are numerous variations and modifications that will be readily apparent to those skilled in the art in light of the above teachings. It is therefore to be understood that, within the scope of the appended claims, the invention may be practiced other than as specifically described.

What is claimed as new and desired to be secured by Letters Patent of the United States is:

1. A flexible body-insertable cable, comprising:
a plurality of conduits, each of said conduits having an antimicrobial material defining interior wall surfaces thereof;
a plurality of cables, each of said cables providing a function selected from the group consisting of mechanical functions, electrical functions, and optical functions; and
a solid antimicrobial material encasing each of said conduits and each of said cables for retaining said conduits and said cables in a spaced-apart and longitudinally-extending arrangement along a length of said solid antimicrobial material.

2. A flexible body-insertable cable as in claim 1, wherein said antimicrobial material defining interior wall surfaces of said conduits comprises a polymeric material impregnated with a biocidal material.

3. A flexible body-insertable cable as in claim 2, wherein said polymeric material is selected from the group consisting of thermoplastic silicone-polyurethane copolymers (“SPUR”), perfluoroethylene propylene copolymer (“FEP”), polytetrafluoroethylene (“PTFE”), thermoplastic polyurethanes (“PUR”), and polyether ether ketone (“PEEK”).

4. A flexible body-insertable cable as in claim 2, wherein said biocidal material comprises cuprous oxide.

5. A flexible body-insertable cable as in claim 1, wherein said solid antimicrobial material encasing each of said conduits and each of said cables comprises a polymeric material impregnated with a biocidal material.

6. A flexible body-insertable cable as in claim 5, wherein said polymeric material comprises perfluoroethylene propylene copolymer (“FEP”).

7. A flexible body-insertable cable as in claim 5, wherein said biocidal material comprises cuprous oxide.

8. A flexible body-insertable cable as in claim 1, further comprising a sheath encasing said solid antimicrobial material encasing each of said conduits and each of said cables, said sheath including a strength member.

9. A flexible body-insertable cable as in claim 8, wherein said sheath comprises polyurethane impregnated with a biocidal material.

10. A flexible body-insertable cable as in claim 1 wherein, for at least one of said conduits, said antimicrobial material defining interior wall surfaces thereof is integral with said solid antimicrobial material encasing each of said conduits and each of said cables.

11. A flexible body-insertable cable, comprising:
a parallel arrangement of conduits and cables;
each of said conduits having an antimicrobial material defining interior wall surfaces thereof;
each of said cables providing a function selected from the group consisting of mechanical functions, electrical functions, and optical functions; and
a solid antimicrobial material encasing said parallel arrangement of conduits and cables.

12. A flexible body-insertable cable as in claim 11, wherein said antimicrobial material defining interior wall surfaces of said conduits comprises a polymeric material impregnated with a biocidal material.

13. A flexible body-insertable cable as in claim 12, wherein said polymeric material is selected from the group consisting of thermoplastic silicone-polyurethane copolymers (“SPUR”), perfluoroethylene propylene copolymer (“FEP”), polytetrafluoroethylene (“PTFE”), thermoplastic polyurethanes (“PUR”), and polyether ether ketone (“PEEK”).

14. A flexible body-insertable cable as in claim 12, wherein said biocidal material comprises cuprous oxide.

15. A flexible body-insertable cable as in claim 11, wherein said solid antimicrobial material encasing said parallel arrangement comprises a polymeric material impregnated with a biocidal material.

16. A flexible body-insertable cable as in claim 15, wherein said polymeric material comprises perfluoroethylene propylene copolymer (“FEP”).

17. A flexible body-insertable cable as in claim 15, wherein said biocidal material comprises cuprous oxide.

18. A flexible body-insertable cable as in claim 11, further comprising a sheath encasing said solid antimicrobial material encasing said parallel arrangement, said sheath including a strength member.

19. A flexible body-insertable cable as in claim 18, wherein said sheath comprises polyurethane impregnated with a biocidal material.

20. A flexible body-insertable cable as in claim 11 wherein, for at least one of said conduits, said antimicrobial material defining interior wall surfaces thereof is integral with said solid antimicrobial material encasing said parallel arrangement.

21. A flexible body-insertable cable, comprising:
a conduit having a first antimicrobial material defining interior wall surfaces thereof;
a plurality of cables, each of said cables providing a function selected from the group consisting of mechanical functions, electrical functions, and optical functions; and
a second antimicrobial material encasing said conduit and said cables for retaining said conduit and said cables in a spaced-apart and longitudinally-extending arrangement along a length of said second antimicrobial material.

22. A flexible body-insertable cable as in claim 21, wherein said second antimicrobial material defines at least one hollow tube along said length of said second antimicrobial material, each said hollow tube being spaced-apart from said conduit and said cables.

23. A flexible body-insertable cable as in claim 21, wherein said first antimicrobial material comprises a polymeric material impregnated with a biocidal material.

24. A flexible body-insertable cable as in claim 23, wherein said polymeric material comprises polytetrafluoroethylene (“PTFE”).

25. A flexible body-insertable cable as in claim 23, wherein said biocidal material comprises cuprous oxide.

26. A flexible body-insertable cable as in claim 21, wherein said second antimicrobial material comprises a polymeric material impregnated with a biocidal material.

27. A flexible body-insertable cable as in claim 26, wherein said polymeric material comprises perfluoroethylene propylene copolymer (“FEP”).

28. A flexible body-insertable cable as in claim 26, wherein said biocidal material comprises cuprous oxide.

29. A flexible body-insertable cable as in claim 21, further comprising a sheath encasing said second antimicrobial material, said sheath including a strength member.

30. A flexible body-insertable cable as in claim 29, wherein said sheath comprises polyurethane impregnated with a biocidal material.
